(19) United States
(12) Patent Application Publication
(10) Pub. No.: US 2004/0034334 A1
(43) Pub. Date: Feb. 19, 2004

(54) Transfer set assembly for multiple lumen catheters

(76) Inventors: Scott Ruddell, Gurnee, IL (US); William Griswold, Bristol, WI (US)

Correspondence Address: BAXTER HEATHCARE CORPORATION, DF3-3E, One Baxter Parkway, Deerfield, IL, 60015, US

(21) Appl. No.: 10/222,969

(22) Filed: Aug. 19, 2002

(51) Int. Cl. A61M 37/00

(52) U.S. Cl. 604/535; 604/6.16

(57) ABSTRACT

A transfer set body for use in transferring dialysis fluid into and out of a patient using an implanted multiple lumen catheter is provided which has a generally cylindrical housing capable of receiving and containing multiple lumen tubing therein, a proximal clamping end which provides pressure so as to pinch off the tubing when it is desired to prevent flow through the tubing, and a distal connection end which allows the tubing to be connected to a source of dialysis fluid and to a drain or other device for collecting or recycling spent dialysate. The transfer set body also features an internal structure of a size and shape which positions the multiple lumen tubing in the body in proper orientation and alignment to allow efficient clamping of the tubing and to ensure that the inflow and outflow lumens are properly aligned.

### FIELD OF THE INVENTION

The present invention relates in general to a transfer set for allowing the safe and secure connection between a catheter from a patient and a source of dialysis fluid and in particular to a transfer set assembly featuring a transfer set body in which multi-lumen tubing may be housed and which features a means for maintaining the proper configuration of the multi-lumen tubing and for clamping off the tubing when desired to prevent flow through said tubing.

### BACKGROUND OF THE INVENTION

Dialysis is a common treatment used to assist patients suffering from a wide variety of kidney problems including severe late stage renal insufficiency which usually results in total or near total kidney failure. This treatment cleans the blood and removes waste products and excess water from the body, a task normally performed by healthy kidneys. Presently, there are two main types of dialysis that are performed to compensate for kidney failure, namely hemodialysis and peritoneal dialysis.

In hemodialysis, the patient's blood is generally passed through a form of artificial kidney in order to cleanse it, followed by the return of the blood to the patient's bloodstream. In peritoneal dialysis, the patient's own peritoneum is used as a semi-permeable membrane in order to remove waste products, and this procedure is carried out by filling the peritoneal cavity with a dialysis solution, also known as dialysate, which preferably is introduced into the body via a permanently implanted catheter. In this process, waste products such as urea and creatinine, as well as excess water, pass from the blood through the peritoneum into the peritoneal dialysis solution, and after a given period of time, or dwell, the used dialysis solution or spent dialysate may be removed from the peritoneal cavity and then either discarded or purified for reuse. In general, the removal of the spent dialysate may be accomplished from a permanent catheter implanted in the body.

Due to the importance of the catheters in carrying out dialysis procedures, it is vitally important that the multiple connections involved in such processes be secure, leak-proof, properly aligned, and easily connectable. This is particularly important using certain catheters which need to be manually connected following implantation into a patient. In addition, since many of these devices are intended for use by a patient on an outpatient basis such as in the home without the supervision of a healthcare professional, it is important that the connections remain secure and in proper position so that there is no leakage or improper delivery of dialysis fluid. It is thus important that such connections be made as simple and effectively as possible so that secure and mistake-proof connections can readily be made manually following implantation of the catheter into a patient. Accordingly, there is a strong need to develop secure, user-friendly catheter connections and connection adapters which can be utilized with dialysis equipment including those units designed for home care on an outpatient basis.

Traditionally, dialysis systems have used catheters with a single lumen wherein the dialysis fluid is introduced into and removed from the patient via the same tube. However, more recently, many dialysis systems now employ multiple lumen catheters wherein it is possible to introduce dialysis fluid through at least one of the lumens and remove the spent dialysate through another lumen in the same catheter. An example of such a multiple lumen system is disclosed in WO 02/30489, incorporated herein by reference, wherein a dual lumen catheter is disclosed in which dialysate flows through a first lumen into the patient and spent dialysate simultaneously flows out through a second lumen. Other multiple lumen catheters have been disclosed for example in patent references including U.S. Pat. Nos. 4,950,259; 5,053,023; 5,167,623; 5,961,485; 5,868,717; 5,683,640; 5,486,159; 5,480,380; 5,380,276; 5,188,593; 5,156,592; 5,057,073; 5,009,636; 5,976,103; 5,961,486; 5,827,237; 5,569,182; 5,221,255; 4,842,582; 4,623,327; and 5,346,471; PCT references WO 94/05363 and 96/29111; European Patent Application 333308; German Patent application 10042067; and Japanese Patent references 8-206216; 2-116380 and 1-303159; all of these patent references incorporated herein by reference.

Although these multiple lumen catheters may increase the efficiency of the dialysis process, they create additional problems with regard to the multiple connections between the dialysis equipment, the lumens of the catheter providing inflow and outflow, and the patient. With such multiple lumen systems, the need to create leak-proof and secure connections is even greater, and it is also important to make the systems mistake-proof and user-friendly so that the proper connections can be made easily and without confusion. It is also necessary to incorporate a transfer that can be easily used by the patient for clamping and unclamping of the catheter system, but these prior multiple lumen catheter systems do not feature such transfer sets.

Similarly, although numerous catheter connection systems are known, few of these even address the particular problems associated with multiple lumen catheter systems, and none provide for a transfer set for safe, efficient and mistake-free use by the patient in peritoneal dialysis systems involving multiple lumen catheters. Examples of such prior systems include those disclosed in patent references including U.S. Pat. Nos. 6,113,572; 4,929,236; 5,743,873; 5,129,891; 5,632,729; 5,399,165; 4,950,255; 5,417,672; 6,190,349; 5,667,490; D431,651; 5,190,529; 5,531,723; 4,781,185; 5,456,676; D303712; 6,254,589; and 6,190,372; Japanese patent references 2000-0045999; 1-171499; and 8-269224; and European Patent EP 553254 B1; all of these patent references incorporated herein by reference.

Another problem with catheters that are implanted in association with their utilization in peritoneal dialysis systems is that some of these catheters, such as the dual lumen catheter disclosed in WO 02/30489, have an implanted section which is made of a relatively hard material such as hard silicone so that they can maintain a proper configuration inside the peritoneal cavity when implanted. However, if such harder material is utilized in the part of the catheter that will be located external to the patient, it can create some discomfort for a patient. Although certain patents relate to catheters having a transitional area with an intermediate durometer, such as U.S. Pat. Nos. 6,135,992; 5,451,206; 5,348,536; 5,125,913; 5,792,124; and 4,739,768, all incorporated herein by reference, none have provided the necessary means whereby a multiple lumen catheter system can be simply and effectively made without such complex and difficult to manufacture materials, and yet provide the necessary internal durometer while maximizing comfort to the patient, being manually connectable, and providing a secure and leak-proof connection.

Further, other prior catheters utilized in procedures such as Continuous Flow Peritoneal Dialysis (CFPD) have had lumens which have a high interference with catheter connection adapter ports used to connect the patient to a source of dialysis fluid, and this makes connection even more difficult to accomplish.

Therefore, in addition to providing a connection assembly for multiple lumen catheters which is user-friendly and mistake-proof, so as to prevent the chance of an erroneous connection, it would be beneficial if the catheter used in connection with this assembly was more comfortable for the patient and could be connected easily with a minimum of resistance or interference.

Finally, in addition to providing a system of connections for multi-lumen catheters which can be made user-friendly and mistake-proof, it is also desirable to develop a system wherein the patient can perform dialysis in the home setting and thus stop or initiate the flow of dialysis fluid into or from the patient's peritoneal cavity, such as during the start or end of a dialysis procedure. Previously, systems wherein the patient could initiate or halt a dialysis operation involving a single-lumen catheter have been known, such as the MiniCap 6 month transfer set produced by Baxter Healthcare Corporation as described in the MiniCap brochure, incorporated herein by reference. However, because this system was specifically designed for use with single lumen catheters, it was not sized or shaped to conduct dialysis fluid from a multiple-lumen catheter, nor did it possess any means for ensuring the proper fluid path or tubing orientation as would be crucial for any devices transferring fluids from a multi-lumen catheter where fluid may be going into and out of the patient at the same time.

Accordingly, it is also desirable to develop a user-friendly transfer set for a multiple lumen catheter by which the user may easily and effectively set up the catheter tubing for dialysis and initiate or stop the flow of dialysis fluid when necessary during a dialysis operation involving a catheter having at least two lumens, i.e., a catheter which will allow both inflow and outflow at the same time.

Therefore, it is important and highly desirable to develop new catheters, catheter connection adapters and patient transfer sets which are user-friendly, mistake proof and which can provide secure and leak-proof connections for systems utilizing multiple lumen catheters, particularly for implanted multiple lumen catheters such as are employed in peritoneal dialysis systems

### SUMMARY OF THE INVENTION

Accordingly, it is an object of the present invention to provide a transfer set assembly to allow the patient to safely and effectively connect an implanted multi-lumen catheter with a source of dialysis fluid.

It is another object of the present invention to provide a transfer set body which is sized and shaped for use with a multiple lumen catheter, and which can be easily unclamped when necessary to start a dialysis procedure and easily clamped and capped when the dialysis procedure is completed.

It is further an object of the present invention to provide a transfer set assembly for use in transferring fluid from a multiple lumen catheter into and out of a dialysis patient which allows the multiple lumen catheter to be properly utilized so as to ensure the correct transfer of fluid into and out of a patient.

It is still further an object of the invention to provide a transfer set body which is easily usable by the patient yet which is flexible enough to be usable with connection adapters or to allow a direct clampable connection between tubing that connects an implanted multiple lumen catheter and the source of dialysis fluid.

It is even further an object of the invention to provide a transfer set body which is sized and shaped to retain multiple lumen tubing used in connecting an implanted multiple lumen catheter to a source of dialysis fluid and to a drain or other means for collecting or recycling spent dialysate, and which has an internal structure capable of maintaining the tubing in proper alignment and in proper position so as to be adequately clamped off when necessary by the transfer set body.

These and other objects are provided by virtue of the present invention which comprises a transfer set body for use in a transfer set assembly for providing access to a source of dialysis fluid and to a means for collecting spent dialysate for a patient having an implanted multiple lumen catheter, said body comprising a generally cylindrical housing capable of receiving and containing therein multiple lumen tubing that is connectable to a multiple lumen catheter implanted in the patient, and having a proximal clamping end comprising a means for compressing said multiple lumen tubing so as to clamp said tubing when it is desired to prevent flow through said tubing and a distal connection end through which a connection will be established from said multiple lumen tubing to a source of dialysis fluid and to a means for collecting spent dialysate, said body having an internal structure which is of a size and shape so as to allow multiple lumen tubing matching said size and shape to be positioned in said body in proper orientation and alignment so that the inflow lumen of the multiple lumen catheter and multiple lumen tubing will be connectable to each other and to a source of dialysis fluid, and the outflow lumen of the multiple lumen catheter and multiple lumen tubing will be connectable to each other and to a means for collecting spent dialysate. The means for clamping the multiple lumen tubing housed in the body of the present invention may comprise any suitable pinching means, such as clamping feet or other suitable clamping device, which are located above and below the tubing and which can be compressed inward at a given location to provide equal pressure to the tubing and pinch off the tubing so as to prevent fluid flow through the tubing. In addition, the internal structure of the transfer set body may comprise any suitable shape which matches up with suitable multiple lumen tubing in such a way that the tubing is in proper alignment and orientation for a peritoneal dialysis operation when housed in the transfer set body. This internal structure may take on a variety of forms, including the use of a slotted opening through the center of the transfer set body wherein the slots line up with matching notches or protrusions located on multiple lumen tubing designed to be housed in the transfer set body of the invention. Still other lock-and-key configurations are possible to ensure that the multiple lumen tubing is properly positioned and aligned in the body so as to be properly clamped when appropriate, and to ensure that inflow takes place only through the inflow lumen, and that outflow occurs only through the outflow lumen.

Through the use of the transfer set body of the present invention, an assembly may be constructed which will allow a patient with an implanted multiple lumen catheter to be safely and properly be connected to a source dialysis fluid and to a means for collecting spent dialysate such as a drain or other means wherein the spent dialysate is recycled or recirculated in some manner. In addition, the transfer set body of the invention allows multiple lumen tubing connected to an implanted multiple lumen catheter to be securely clamped so that flow is prevented until such time as it is desired to initiate a dialysis operation, at which point the patient may safely and correctly connect the transfer set body to a source of dialysis fluid, unclamp the device, and allow dialysis to take place. Following completion of the dialysis procedure, the patient may readily clamp the tubing once again, disconnect the tubing from the source of dialysis fluid, and recap the transfer set body until needed again. The transfer set body thus ensures that the tubing will stay clamped between dialysis operations, and thus serves as a secondary seal during this period so as to further prevent infections.

These embodiments and other alternatives and modifications within the spirit and scope of the disclosed invention are described in, or will become readily apparent from, reference to the detailed description of the preferred embodiments provided herein below.

### BRIEF DESCRIPTION OF THE DRAWING FIGURES

FIG. 1 is a perspective view of the transfer set body of the present invention.

FIG. 2 is a perspective view of the transfer set body of the present invention in combination with multiple lumen tubing housed in the transfer set body.

FIG. 3 is perspective view of the transfer set body of the present invention in combination with a sleeve capable of clamping multiple lumen tubing when housed in the transfer set body.

FIG. 4 is a side, partially cutaway view showing the transfer set body of the invention in combination with a sleeve in clamped position as shown in FIG. 3.

FIG. 5 is a perspective view of a transfer set clamping sleeve usable with the transfer set body of the present invention.

FIG. 6 is a prespective view of the combination of the transfer set body, sleeve and multiple lumen tubing in accordance with the invention in combination with a threaded connection adapter.

FIG. 7 is a perspective view of a cap usable with the transfer set body of the present invention.

### DETAILED DESCRIPTION OF THE PREFERRED EMBODIMENTS

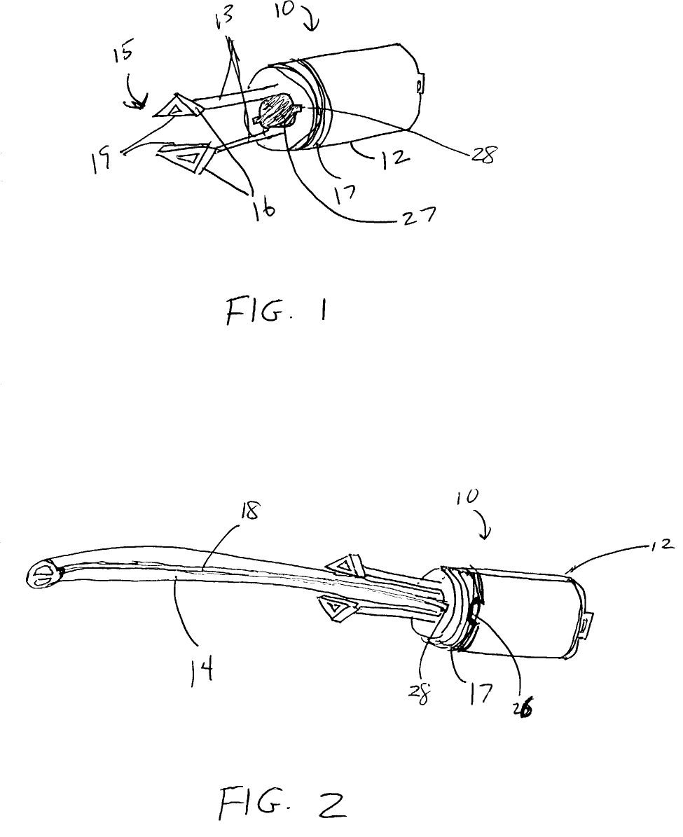

In accordance with the present invention, there is provided a transfer set body for providing a conduit for dialysate fluid into and out of a patient using an implanted multiple lumen catheter which emerges from the patient and which must be connected properly to both a source of dialysis fluid and to a collecting device, drain or other means capable of receiving and/or recycling or regenerating spent dialysate from the patient that is generated during a dialysis procedure. As best observed in FIGS. 1 and 2, the transfer set body 10 of the invention comprises a generally cylindrical housing 12 capable of receiving and housing multiple lumen tubing 14 therethrough, and having a clamping means which comprises a means for compressing the tubing in at least one given location in a manner wherein generally equal compression is applied inwardly at the top and bottom of the tubing so as to pinch off the tubing when it is desired to prevent flow. This compression means can take on a variety of forms, including clamping feet, a roller and ramp system, pinching claws from the top and bottom of the transfer set body, etc. In one particular embodiment, as shown in FIGS. 1 and 2, the body 10 has a proximal clamping end 15 comprising at least two opposing clamping feet 16 which are capable of being compressed inward so as to pinch off multiple lumen tubing 14 when disposed in said body 10. These clamping feet 16 are positioned above and below said tubing 14 so that when brought together, they pinch off the tubing so as to function as a clamp and restrict or stop the flow of fluid through said tubing. In the preferred embodiment, the feet 16 extend outward from a central portion 17 of housing 12 by means of prongs 13 disposed at the general periphery of housing 12, and preferably at the top and bottom, respectively, of opening 27 of central portion 17 which is designed to receive multiple lumen tubing 14.

The clamping feet 16 may have any suitable size and shape that will permit the feet to be brought down upon the multiple lumen tubing 14 in body 10, and will generally have a flat surface 19 so as to apply equal pressure from the top and bottom of the multiple lumen tubing. As shown in the embodiment of FIGS. 1 and 2, these clamping feet 16 may be configured as triangular in shape with the flat ends 19 facing each other and being generally parallel to the horizontal axis of tubing 14 when properly positioned in the transfer set body 10. Once again, this design is preferred so that the two flat surfaces 19 of clamping feet 16 will be pressed down simultaneously when compressed so as to apply equal pressure from top and bottom and ultimately provide a sufficient clamping action so as to pinch off or clamp tubing 14.

Although shown in the embodiment with triangular-shaped clamping feet, the invention contemplates that the clamping feet may be of a large variety of shapes and forms which are suitable for compression around the multiple lumen tubing 14 so that the tubing is pinched off at the location where the clamping feet apply pressure to the tubing. In addition, as described above, the compression means of the invention may take on a variety of other forms such as a roller clamp (not shown) which has a rigid lower ramp and a roller arm, and the transfer set body may be clamped when necessary to prevent flow by rolling the roller arm over the rigid ramp toward the proximal clamping end until proper compression and clamping occurs on the multiple lumen tubing. Other suitable forms include any other means capable of providing compression of the tubing to prevent flow in at least one given location on the tubing, and may include pinching projections from the top and bottom of the body which can be brought down on the tubing to prevent flow. In addition, the body 10 can be suitably sized and shaped to receive multiple lumen tubing in a variety of sizes and shapes, e.g., wherein the lumens have a double-D configuration, a double oval configuration, or even where such tubing has separate lumens. In all of these cases, the body 10 will be suitably designed to match said configurations and maintain the multiple lumen tubing in proper position and alignment for transferring fluid to and from a patient during a dialysis procedure.

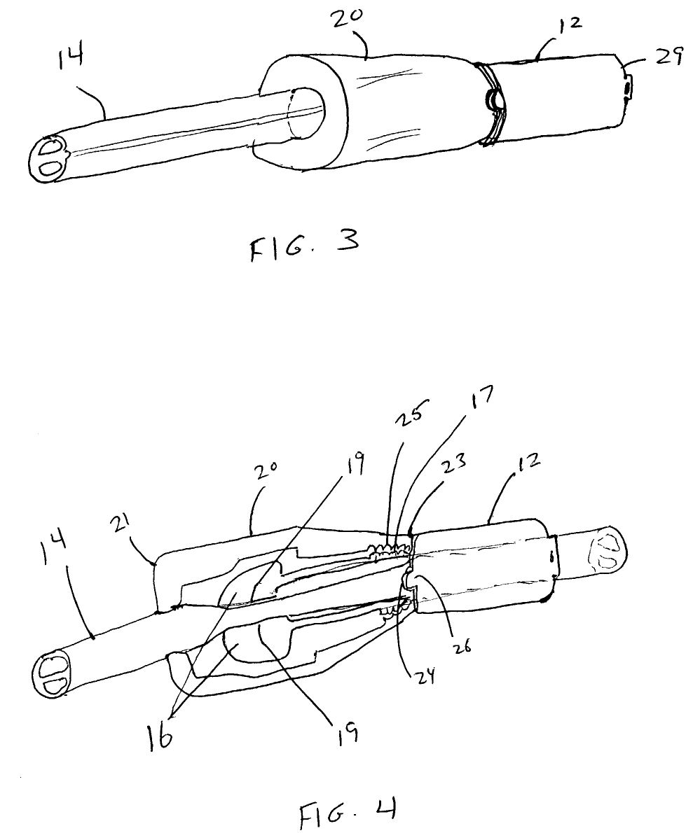

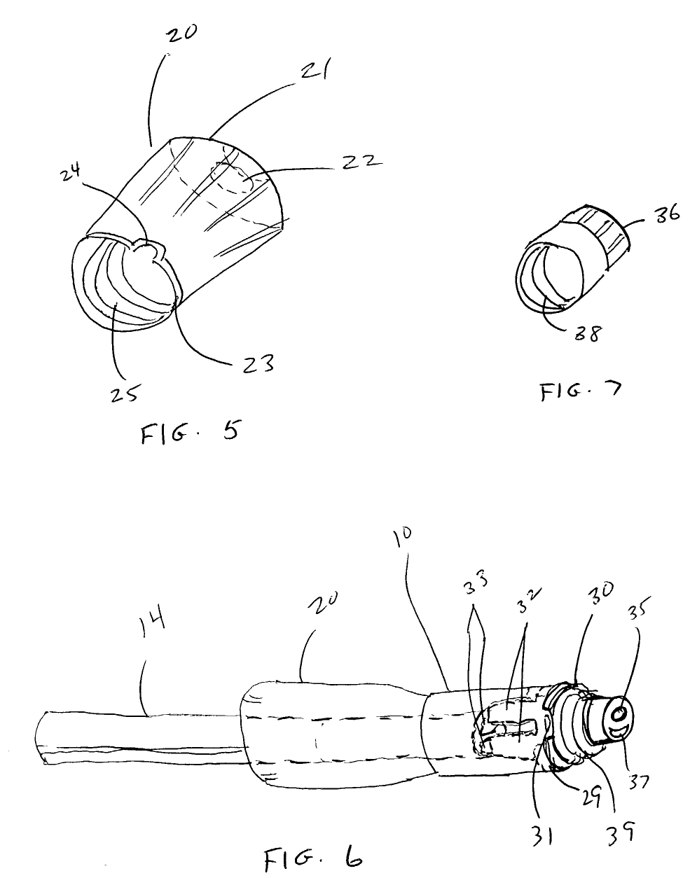

In the embodiment as shown in Figures and 2, it is preferable that the transfer set body 10 of the invention be utilized in combination with a suitable transfer set clamping sleeve 20, as best observed in FIGS. 3-5. As shown in these drawing figures, the transfer set sleeve 20 is preferably sized to fit snugly over the clamping feet 16 of transfer set body 10, and when positioned over said clamping feet causes those feet to be compressed inward over tubing 14 so as to seal off the tubing. In the preferred embodiment, the sleeve 20 is generally cylindrical in shape and will be sized so as to fit over the clamping feet 16 at proximal clamping end 15 of the body 10. In addition, the sleeve 20 will preferably include an opening 22 at the proximal end 21 of the sleeve, and this opening is itself sized to fit properly around multi-lumen tubing 14. At the other end 23 of sleeve 20 which is the end designed to be connected directly to housing 12 of transfer set body 10, it is preferred that the internal surface 25 be threaded so as to allow the sleeve to simply be screwed into place on body 10 when clamping is desired. In the embodiment wherein the suitable clamping sleeve 20 will be threaded, it is thus preferred that the transfer set body 10 be threaded at a central portion 17 so as to allow the sleeve 20 to be screwed or twisted into place onto the housing 12 of transfer set body 10 when clamping is desired. In a particularly preferred embodiment, the sleeve 20 also contains a notch 24 at end 23 which will fit snugly in place with matching nub 26 at the central portion 17 of sleeve 10. This nub and notch arrangement will assist in maintaining the sleeve in the clamped position when so desired, such as between dialysis procedures. Further, another preferred feature of the sleeve 20 is that it is tapered inward from the outer end 21 to the inward end 23, as best shown in the side, partially cutaway view of FIG. 4, and once again this tapered design is utilized to assist in the proper clamping of tubing 14 when the sleeve 20 is brought around the transfer set body 10 so as to achieve compression of clamping feet 19 around tubing 14. In operation, the sleeve is initially in its twisted and clamped position, but can be opened by the dialysis patient when necessary to initiate a dialysis procedure, and then simply reclosed by twisting when the dialysis procedure is completed.

Another feature of the transfer set body 10 of the present invention is the provision of an internal structure which is sized and shaped to receive multi-lumen tubing such as tubing 14 and which ensures that the tubing will be maintained in the correct position to ensure proper inflow and outflow of the dialysis fluid when an implanted multiple lumen catheter is used in a continuous peritoneal dialysis procedure. In addition, as indicated above, the internal structure is designed so as to be compatible with the clamping means and ensure proper positioning of the multiple lumen tubing housed in the transfer set body so that it can be adequately clamped when so desired by the patient. This internal structure can take on a variety of forms but will generally be designed so that a particular configuration of the internal structure will match the configuration of the multiple lumen tubing designed to be used with transfer set body 10 in such a way that the tubing will be properly positioned in body 10 to ensure orientation and alignment wherein the inflow paths will only be connected to the source of dialysis fluid and the outflow paths will only be connected with the means for connecting spent dialysate. The internal configuration of body 10 will also ensure that the tubing 14 is properly positioned so that suitable clamping can occur in at least one given location via the compression means of the transfer set body 10. Accordingly, this internal structure may take on a variety of forms suitable to ensure the proper positioning of at least the inflow and outflow lumens of the multiple lumen tubing that will be housed in body 10, including arrangements wherein the internal structure will have slots or other openings that match up with notches or protrusions in the multiple lumen tubing. Still other forms wherein the tubing and internal structure match up in lock and key fashion to ensure proper positioning and alignment in body 10, and these include the lock-and-key configurations described with regard to the connecting devices as disclosed in co-pending U.S. patent application Ser. No. 10/_______ of Seese et al., relating to catheter connection adapters, said application being incorporated herein by reference.

In one particular embodiment, as shown for example in FIGS. 1 and 2, the internal configuration of body 10 may comprise a slotted opening 27 that is preferably sized to match the exterior surface of a multi-lumen tube 14, and will preferably possess at least one notch or slot 28 at the midportion of opening 27 which will match a similarly sized and shaped rib or ridge 18 on the multiple lumen tubing 14. The slot or notch 28 of body 10 and the rib or ridge 18 of tubing 14 are preferably located at the horizontal midpoint between upper and lower clamping feet 22, so as to form a central protruding ridge 18 which fits into a central slot 28, and this slot/rib feature is designed so that the multiple lumen tubing 14, which is connected to the implanted multiple lumen catheter coming from the patient, is properly positioned in transfer set body 10 with the tubing's upper and lower lumen matching the inflow and outflow lines of the source for dialysis fluid utilized in a dialysis operation. In addition, this slot/rib feature also allows the tubing to be properly oriented so that the clamping feet 16 can accurately pinch off the multiple lumen tubing using equal pressure from top and bottom to ensure adequate closure of the tubing when so desired. When so desired, the slot or notch in opening 27 may be provided on both sides of the opening and in this case, the notches are designed to match the external profile of ribs or ridges on both sides of tubing 14. The opening 27 is shown at the central portion of body 10 but may be positioned at any suitable location, e.g., at the proximal end or distal end of body 10 as well.

As indicated above, other forms which ensure proper positioning and alignment of the multiple lumen tubing 14 on body 10, such as other geometric configurations of the tubing and internal body structure which fit in lock-and-key fashion, are also contemplated with regard to the present invention. In general, any form of the internal structure which is of a size and shape so as to allow multiple lumen tubing matching that size and shape to be positioned in transfer set body 10 in proper orientation and alignment so that the inflow lumen of the multiple lumen catheter and multiple lumen tubing will be connectable to each other and to a source of dialysis fluid, and the outflow lumen of the multiple lumen catheter and multiple lumen tubing will be connectable to each other and to a means for collecting spent dialysate, will be suitable for use in the invention.

In the preferred embodiment, the transfer set body 10 is thus employed in a transfer set assembly which will connect a patient having an implanted multiple lumen catheter to both a source of dialysis fluid and to a drain means for collecting and/or recycling or regenerating spent dialysate. In addition, the transfer set assembly using body 10 will allow the patient to readily connect multiple lumen tubing housed in body 10 to a source of dialysis fluid when it is desired to initiate a peritoneal dialysis operation, at which point the compression means such as clamping feet 16 may be opened by unscrewing clamping sleeve 20 to allow the flow of dialysis fluid into the patient. At the same time, when a continuous flow peritoneal dialysis procedure is utilized, inflow of the dialysis fluid may occur simultaneously with the outflow of spent dialysis fluid from the patient to a suitable collecting or recycling means. When the operation is completed, the patient may apply the compression means so as to prevent flow by clamping off tubing 14, and the patient may then disconnect the multiple lumen tubing from the source of dialysis fluid and/or the means for collecting spent dialysate. In the preferred embodiment, the multiple lumen tubing 14 which is retained in the transfer set body 10 and which can be clamped using clamping feet 16 will be connected to a source of dialysis fluid at end 29 of body 10 in any of a number of suitable ways. As will be described below, it is possible to utilize a connection adapter which will fit into the body 10 at end 29 and will contain ports for transferring inflow and outflow dialysis fluid travelling through tubing 14. However the tubing 14 is ultimately connected to a source of dialysis fluid, it normally will be capped when not in use, such as by a suitable sterile plastic cap 36 which may be capable of applying an aseptic material to the end of the tubing 14 after completion of a dialysis procedure.

As indicated above, the transfer set body 10 of the invention will allow the connection between a multiple lumen catheter that has been implanted into a patient and a source of dialysis fluid. In this respect, the tubing 14 that enters from the patient side of the body 10 will be connected the source of dialysis fluid at the other end 19 of the body 10. In order to assist in the transfer of fluid from the end 29 of body 10 to the dialysate source, a connection adapter 30 may be employed which can fit inside end 29 of transfer set body 10, such as shown in FIG. 6. This connection adapter 30 includes at least two ports 32 which will fit snugly into the lumens 33 of tubing 14 and will allow passage of the fluid through at least an inflow lumen, which carries fluid from the source of dialysis fluid and into the patient, and at least one outflow lumen which will transfer spent dialysate from the patient to a suitable drain or other receiving means. The connection adapter 30 also will include openings for transmitting fluid to and from tubing 14, such as an opening 35 at the center of the adapter 30 and an opening 37 at the periphery of adapter 30. Again, these openings are designed so that at least one opening can be used for transmitting fluid into a patient, and at least one opening can be used to transfer spent dialysate out of a patient. In the embodiment wherein transfer set body 10 will be used with the connection adapter 30, the transfer set body preferably contains means, such as slot arrangement 31 which will act to receive a mating portion around the external surface of connection adapter 30 so as to lock the connection adapter in place. Similarly, the connection adapter may contain external threading at the point where it is inserted into end 29 of body 10, and the body 10 may have internal threading which matched threading from the adapter so that the adapter may be screwed in place in transfer set body 10 if so desired.

As also shown in FIGS. 6 and 7, the transfer set body 10 of the invention will preferably be used with a cap 36 which will be placed over the end 29 of body 10 so as to cap off the tubing 14 and provide an aseptic cover to protect against infection when the transfer set body is not in use. In the preferred embodiment, cap 36 will also contain a prepackaged amount of an aseptic material that will be applied when the transfer set body 10 is capped off for purposes of disinfecting the system and protecting against further infection. In the preferred embodiment, as shown in FIGS. 6 and 7, the cap has internal threading 38 to assist in affixing the cap to the transfer set body 10 or to the connection adapter 30, if so provided, at end 29 of transfer set body 10. In the embodiment as shown in FIG. 6, the connection adapter 30 contains an external threaded area 39 adjacent the point where it attaches to the end 29 of transfer set body 10, and this threaded area 39 will match up with the internal threading 38 of cap 36 so that the cap may be easily threaded onto the adapter 30 when so desired by the patient. When put into place and connected with a suitable multiple lumen catheter and multi-lumen tubing, the transfer set body 10 will thus be capped until it is desired to initiate a dialysis procedure. When dialysis needs to be initiated, the cap may be removed by the patient, and the transfer set body is connected to a source of dialysis fluid, such as through connection adapter 30, and is also connected to a means, such as a drain, for receiving or collecting spent dialysate. Following the completion of a peritoneal dialysis procedure, the transfer set body is clamped, then disconnected from the source of dialysis fluid, and is recapped preferably by screwing aseptic cap 36 onto the connection adapter 30 connected to end 29 of body 10.

In the use of the transfer set body of the present invention, there is thus a wide range of adapters and systems that can be utilized in order to connect a patient to a source of dialysis fluid. In addition to the connection adapter as described above, a patient connector (not shown) may be used which screws onto the connection adapter and provides a connection both to a source of dialysis fluid and to a means for receiving spent dialysate. In such a system, the patient connector will preferably contain internal threading so as to allow it to be screwed onto the threading 39 of connection adapter 30, and this system will allow a patient to initiate a dialysis operation by removing cap 36, connecting the connection adapter 30 to a patient connector, and then untwisting sleeve 20 around the clamping feet 16 of transfer set body 10 which unclamps tubing 14 and allows a peritoneal dialysis operation to take place. At the conclusion of the dialysis procedure, the twist sleeve 20 is twisted back onto clamping feet 16 so as to pinch off tubing 14, and the patient connector may be removed from connection adapter 30, and the cap 36 is then replaced onto the connection adapter to apply an aseptic material and to disinfect the system and to protect the transfer set body from infection while not in use.

As would be apparent to one of ordinary skill in the art, the transfer set body and assembly as described above can be made from a variety of sturdy, sterilizable materials commonly used in dialysis devices. For example, the transfer set body of the invention may be manufactured of any suitable rigid, chemical and creep resistant plastic which should also be lightweight yet sturdy, sterilizable and injection moldable. Any of a number of commercially available plastics are suitable for use in the present invention including polycarbonate, polyurethane, or other suitable hard plastic. Similarly, the twist sleeve, connection adapter and cap that may be used in conjunction with the transfer set body may also be made of any suitable sterilizable plastic material commonly used in devices utilized in peritoneal dialysis systems. In addition, the transfer set body of the invention can be utilized in systems employing the catheter connectors as disclosed in co-pending U.S. patent application 10/______ of Seese et al., and in systems utilizing the multiple lumen catheters as disclosed in co-pending U.S. patent application 10/______ of Seese et al., both applications being incorporated herein by reference.

As indicated above, the present transfer set body is thus advantageous in that it allows for the first time a simple, mistake-proof transfer device that may be utilized with an implanted multi-lumen catheter and a multi-lumen tubing system connected to said catheter, and which provides a simple system that is user-friendly and which can be easily operated by the patient when it is desired to initiate dialysis, and readily clamped and capped off in a safe and aseptic manner when the dialysis procedure is completed.

The present invention has been described above with regard to exemplary embodiments, but as will be understood by those of ordinary skill in the art, the present invention encompasses numerous additional embodiments which will fall within its scope in addition to the specific embodiments described above.

What is claimed is:

1. A transfer set body for use in an assembly for providing access to a source of dialysis fluid and to a means for collecting spent dialysate for a patient having an implanted multiple lumen catheter, said body comprising a generally cylindrical housing capable of receiving and containing therein multiple lumen tubing that is connectable to a multiple lumen catheter implanted in the patient, and having a proximal clamping end comprising a means for compressing said multiple lumen tubing so as to clamp said tubing when it is desired to prevent flow through said tubing and a distal connection end through which a connection will be established from said multiple lumen tubing to a source of dialysis fluid and to a means for collecting spent dialysate, said body having an internal structure which is of a size and shape so as to allow multiple lumen tubing matching said size and shape to be positioned in said body in proper orientation and alignment so that the inflow lumen of the multiple lumen catheter and multiple lumen tubing will be connectable to each other and to a source of dialysis fluid, and the outflow lumen of the multiple lumen catheter and multiple lumen tubing will be connectable to each other and to a means for collecting spent dialysate.

2. The transfer set body as claimed in claim 1 wherein the compression means comprises at least two opposing clamping feet which are capable of being compressed inward around multiple lumen tubing disposed in said body so as to clamp off said tubing when necessary.

3. The transfer set body as claimed in claim 2 wherein said clamping feet extend outward from the central portion of said body.

4. The transfer set body as claimed in claim 2 wherein the clamping feet are in the form of two appendages each with a flat end, one end positioned above tubing when housed in the transfer set body, and one end positioned below said tubing so that when said appendages are compressed inward, the flat ends will impinge upon and pinch off said tubing.

5. The transfer set body as claimed in claim 1 wherein the compression means comprises a ramp and a roller arm capable of rolling onto said ramp in the direction towards the proximal clamping end of said body so as to achieve compression of the tubing at a given location and clamp off said tubing when necessary.

6. The transfer set body as claimed in claim 1 wherein the internal structure of said body comprises a slotted opening which is of a size and shape so as to allow multiple lumen tubing having a notch or protrusion which matches the slot in the opening in the internal structure to be positioned in said device in proper orientation for conducting a peritoneal dialysis operation using a multiple lumen catheter that has been implanted in a patient.

7. The transfer set body as claimed in claim 6 wherein said slotted opening is located in a central portion of said transfer set body.

8. The transfer set body as claimed in claim 6 wherein said slotted opening is located at one of the ends of said transfer set body.

9. The transfer set body as claimed in claim 6 wherein the slotted opening is in the form of a generally circular opening having at least one central notch along the horizontal axis of said body which will match with a ridge along the side of the tubing to be housed in said body so that said tubing will be housed in said body in proper orientation to transfer fluid into and out of the patient having an implanted multiple lumen catheter, and to ensure proper clamping of the multiple lumen tubing when so desired.

10. The transfer set body as claimed in claim 9 wherein the circular opening has notches along both sides of the horizontal axis which match the external profile of side ridges of the tubing to be housed in said body.

11. The transfer set body as claimed in claim 1 wherein the internal structure is of a configuration which mates in lock and key fashion with matching multiple lumen tubing to ensure proper orientation and alignment of said tubing when housed in said body.

12. The transfer set body as claimed in claim 1 wherein the outer surface of said body at a point between the proximal and distal end is threaded so as to receive a sleeve which will be screwed onto said body at said proximal clamping end so as to permit compression of the tubing housed in said body when it is desired to prevent flow through said tubing.

13. The transfer set body as claimed in claim 1 wherein said distal connection end is slotted so as to receive a mating connection adapter capable of connecting the multiple lumen tubing housed in said body with both a source of dialysis fluid and a means for collecting spent dialysate coming from the patient.

14. The transfer set body as claimed in claim 1 in combination with a sleeve that can be moved onto the proximal clamping end of said body so as to cause compression and achieve clamping of the tubing housed in said body.

15. The transfer set body as claimed in claim 14 wherein said sleeve is a twist sleeve that can be screwed onto the proximal clamping end of said body so as to cause compression and achieve clamping of the tubing housed in said body.

16. The transfer set body as claimed in claim 2 in combination with a sleeve that can be moved onto the proximal clamping end of said body so as to cause compression of the clamping feet and achieve clamping of the tubing housed in said body.

17. The transfer set body as claimed in claim 1 in combination with multiple lumen tubing housed in said body.

18. The transfer set body as claimed in claim 17 wherein said multiple lumen tubing is connected to a multiple lumen catheter implanted in a patient in need of dialysis treatment.

19. The transfer set body as claimed in claim 18 wherein the multiple lumen tubing is connected to the multiple lumen catheter implanted in the patient by means of a catheter connector.

20. The transfer set body as claimed in claim 1 wherein the multiple lumen tubing has at least two lumens.

21. The transfer set body as claimed in claim 1 wherein the multiple lumen tubing is dual lumen tubing.

22. The transfer set body as claimed in claim 1 in combination with a connection adapter which can be connected to said body and which can connect multiple lumen tubing housed in said body with a source of dialysis fluid.

23. The transfer set body as claimed in claim 22 wherein the connection adapter has external threading which is capable of receiving a cap.

24. The transfer set body as claimed in claim 22 in combination with a cap.

25. The transfer set body as claimed in claim 1 in combination with a cap
